(19) United States
(12) Patent Application Publication
(10) Pub. No.: US 2026/0108681 A1
(43) Pub. Date: Apr. 23, 2026

(54) Pinching Clip for Subcutaneous Injections

(71) Applicant: Nicholas Demes Owens, Sarasota, FL (US)

(72) Inventor: Nicholas Demes Owens, Sarasota, FL (US)

(21) Appl. No.: 18/918,468

(22) Filed: Oct. 17, 2024

(51) Int. Cl. A61M 5/42

(52) U.S. Cl. CPC A61M 5/425

(57) ABSTRACT

A skin-pinching device for facilitating subcutaneous injections, particularly in difficult-to-reach areas such as the skin around the elbow, is disclosed. The device comprises a first and second handle connected at a pivot, and a first and second pinching end pivotally connected to the handles. The pinching ends feature a plurality of spikes and are coated with a high-friction material, such as rubber or polymer, to enhance grip on the skin. When the handles are pressed together, the pinching ends move away from each other, allowing the device to be placed over a section of skin to be pinched. Upon releasing the handles, the pinching ends move towards each other, pressing against the skin to securely grip the section of tissue. The handles are positioned to provide sufficient space for needle insertion between the pinching ends when the device is gripping the skin.

### FIELD OF THE DISCLOSURE

The present invention relates to the field of medical devices, specifically to assistive devices for administering subcutaneous injections. More particularly, the invention pertains to a pinching clip with spiked gripping surfaces that enables users to securely pinch and hold skin with one hand, facilitating single-handed injections in difficult-to-reach areas, such as the skin around the elbow.

### BACKGROUND OF THE INVENTION

Subcutaneous injections are a common method of administering medications, particularly for individuals with chronic conditions requiring frequent or daily injections. These injections involve delivering medication into the fatty tissue beneath the skin using a short needle. While subcutaneous injections are generally considered safe and effective, they can be challenging for some patients to administer, especially when targeting difficult-to-reach areas such as the skin around the elbow.

Traditionally, patients have been taught to use the “pinch method” when administering subcutaneous injections. This technique involves pinching the skin with one hand to create a fold of tissue, allowing for easier needle insertion and ensuring that the medication is delivered into the subcutaneous layer. However, this method requires the use of both hands, making it difficult for patients to self-administer injections in certain areas of the body.

Attempts have been made to address this issue, with some patients resorting to using makeshift devices like “chip clips” to pinch the skin, freeing one hand for injection. While these improvised solutions may provide some assistance, they have significant limitations. Chip clips and similar devices often have a limited opening range, making it difficult to pinch an adequate amount of skin. Additionally, the smooth, hard surfaces of these clips do not provide sufficient grip on the skin, increasing the risk of slippage during the injection process.

Furthermore, the fear and anxiety associated with needles and injections can make the process more challenging for some patients. Desensitizing devices that use soft spikes to stimulate the nerves around the injection site have been developed to help alleviate these concerns. However, these devices are separate from the skin-pinching tools, requiring patients to use multiple devices during the injection process.

The object of the present invention is to provide a purpose-built, easy-to-use device that enables patients to securely pinch and hold skin with one hand while administering subcutaneous injections, particularly in difficult-to-reach areas such as the skin around the elbow, improving the overall experience and effectiveness of the injection process.

### SUMMARY

The following summary provides an overview of some of the key inventive features of the skin-pinching device for facilitating subcutaneous injections. This summary is not an extensive overview of the invention and is not intended to identify all key or critical elements of the invention or to delineate the scope of the invention. Its sole purpose is to present some concepts of the invention in a simplified form as a prelude to the more detailed description that is presented later.

In some aspects, the present invention relates to a skin-pinching device designed to facilitate subcutaneous injections. The device consists of a first handle and a second handle connected at a pivot, with a first pinching end pivotally connected to the first handle and a second pinching end pivotally connected to the second handle. Both pinching ends feature a plurality of spikes, which, in certain embodiments, may be coated with a high-friction material, such as rubber or polymers, to enhance grip on the skin.

One aspect of the invention describes a device that enables users to administer subcutaneous injections using one hand while the device holds the pinched skin in place. When the first and second handles are pressed together, the first and second pinching ends move away from each other, allowing the device to be placed over a section of skin to be pinched. Upon releasing the handles, the pinching ends move towards each other, pressing against the skin to securely grip the section of tissue.

In certain embodiments, the device features a pivot mechanism that provides sufficient space for needle insertion between the first and second pinching ends when the handles are squeezed together, facilitating easy and accurate injection. The pivot may also include a mechanism for self-closing the pinching ends when the handles are released, ensuring a secure grip on the skin throughout the injection process.

In further aspects, the skin-pinching device may allow for the needle to be inserted into the pinched section of skin from either side of the pinching ends, providing flexibility and convenience for the user. Further, securely gripping the skin and desensitizing the injection site, the device reduces pain and feelings of anxiety associated with subcutaneous injections, making the process more comfortable and less daunting for users.

Some aspects of the invention describe substantially flattened sections on the first and second pinching ends, offering a stable and comfortable gripping surface for the skin. In certain embodiments, the pivot may feature a substantially enlarged through hole or a C-shaped curve between the first and second handles, allowing for easy passage of the needle during injection.

Another aspect of the invention involves detachable and replaceable first and second pinching ends, allowing for easy cleaning or replacement of the spiked surfaces, ensuring optimal hygiene and performance over time. The inventive skin-pinching device streamlines the subcutaneous injection process, particularly in difficult-to-reach areas, by enabling users to pinch and hold skin with one hand while administering injections with the other, ultimately improving the ease, accuracy, and comfort of the injection experience.

### BRIEF DESCRIPTION OF THE DRAWING

FIG. 1 is a perspective view of a skin-pinching device according to one aspect of the invention.

FIG. 2 illustrates the skin-pinching device attaching to a skin section of the hand.

FIG. 3 shows a needle injecting a pinched skin through a space between the first and second pinching ends.

FIG. 4 is a close-up view of the pinching device attaching to the skin, raising a section to be injected.

FIG. 5 illustrates a needle passing between the first and second handles of the pinching device.

FIG. 6 is a close-up top view of the pinching device in an open position, showing a spring that keeps the pinching ends close together.

FIG. 7 illustrates a rounded construction of the pinched end.

FIG. 8 illustrates a substantially square construction of the pinched end.

### DETAILED DESCRIPTION OF THE INVENTION

As used herein, the terms “comprising,” “including,” “containing,” “characterized by,” and grammatical equivalents thereof are inclusive or open-ended and do not exclude additional, unrecited elements or method steps, unless otherwise stated. Other than in the operating examples, or where otherwise indicated, all numbers expressing measurements, dimensions, and so forth used in the specification and claims are to be understood as being modified in all instances by the term “about,” meaning within a reasonable range of the indicated value.

The present invention provides a novel skin-pinching device for facilitating subcutaneous injections, aimed at improving safety, comfort, and user experience. The inventive device comprises specific structural elements and features arranged in a defined configuration, designed to address the limitations of existing skin-pinching methods and devices. The following detailed description discloses various embodiments, aspects, and features of the present invention, which are not intended to limit the scope of the invention in any way but rather to exemplify the preferred embodiments.

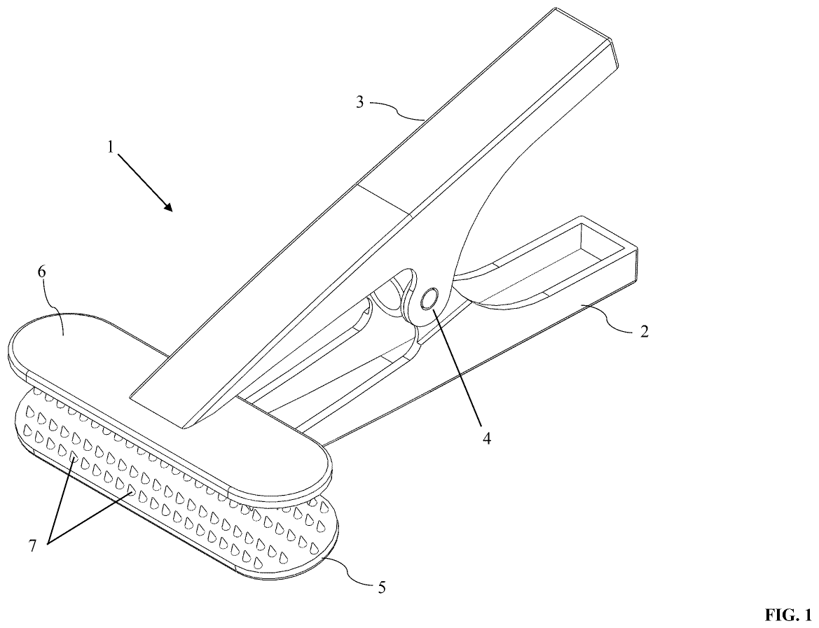

The FIG. 1 is a perspective view of a skin-pinching device 1 according to one aspect of the invention. The device comprises a first handle 2 and a second handle 3, which are connected at a pivot 4. The pivot 4 allows the first handle 2 and second handle 3 to move relative to each other.

Attached to the first handle 2 is a first pinching end 5, which is pivotally connected to the handle. Similarly, a second pinching end 6 is pivotally connected to the second handle 3. The first pinching end 5 and second pinching end 6 are positioned opposite each other and are designed to move towards or away from each other as the first handle 2 and second handle 3 are moved.

In this embodiment, both the first pinching end 5 and second pinching end 6 feature a plurality of spikes 7 on their surfaces. These spikes 7 may be arranged in a pattern that covers a substantial portion of the pinching ends'surfaces. Preferably, the spikes 7 are shown as small, pointed protrusions that extend outwardly from the pinching ends.

The first handle 2 and second handle 3 are shown as elongated, preferably but not necessarily curved members that are designed to be comfortably gripped by a user's hand. The pivot 4 connecting the handles is depicted as a cylindrical structure that allows for smooth rotation of the handles relative to each other.

Preferably, the skin-pinching device 1 in FIG. 1 may be a compact, handheld tool with a symmetrical design. The pivoting action of the handles and pinching ends, along with the presence of spikes on the pinching surfaces, allow the device to grip or pinch a targeted area securely.

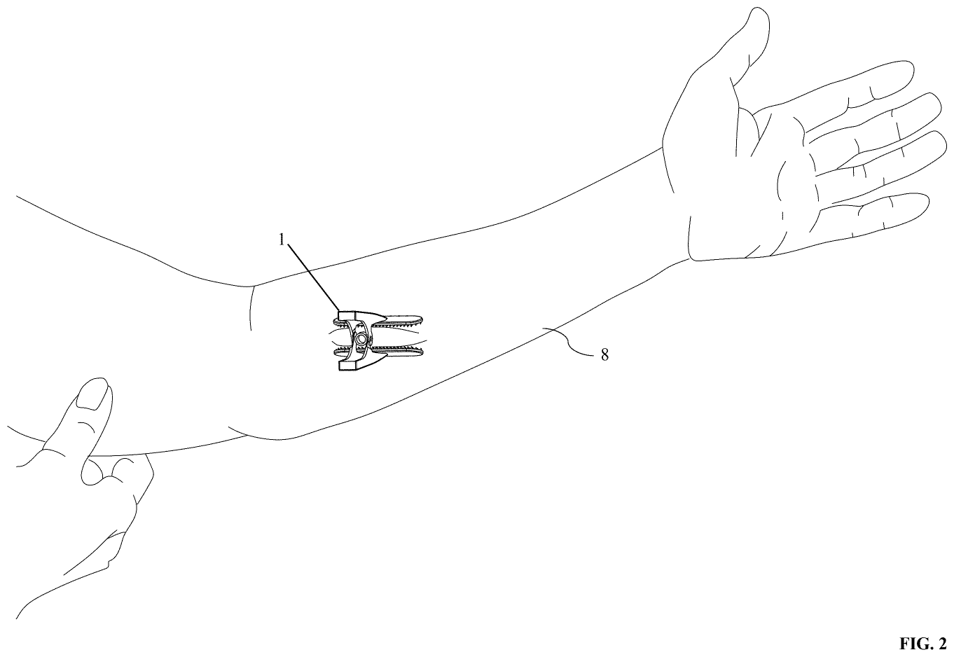

The exemplary embodiment of FIG. 2 illustrates the skin-pinching device 1 in use, attached to a section of skin on a hand 8. The device is shown gripping a pinched portion of skin, demonstrating its primary function of securely holding a targeted area for subcutaneous injection.

In this figure, the skin-pinching device 1 is depicted with its first pinching end and second pinching end closed together, firmly grasping the skin on the back of the hand 8. The plurality of spikes on the pinching ends are pressed against the skin, creating a localized area of pressure.

The spikes serve a unique purpose in desensitizing the injection site. By overloading the nerves in the targeted area, the spikes help to reduce the sensation of the impending injection. This desensitization effect is achieved through the concentrated stimulation of the skin's surface, which temporarily diminishes the perception of pain or discomfort.

As the skin-pinching device 1 maintains its grip on the pinched skin, it creates a stable and secure platform for administering a subcutaneous injection. The device's handles are shown extending outwardly from the pinched area, providing ample space for the user to hold and manipulate the device with one hand while preparing the injection with the other.

The hand 8 in FIG. 2 is represented in a relaxed position, with the skin-pinching device 1 attached to the dorsal aspect of the hand. The device's compact size and ergonomic design allow it to be easily positioned and used on various parts of the body, including difficult-to-reach areas such as the back of the hand or the skin around the elbow.

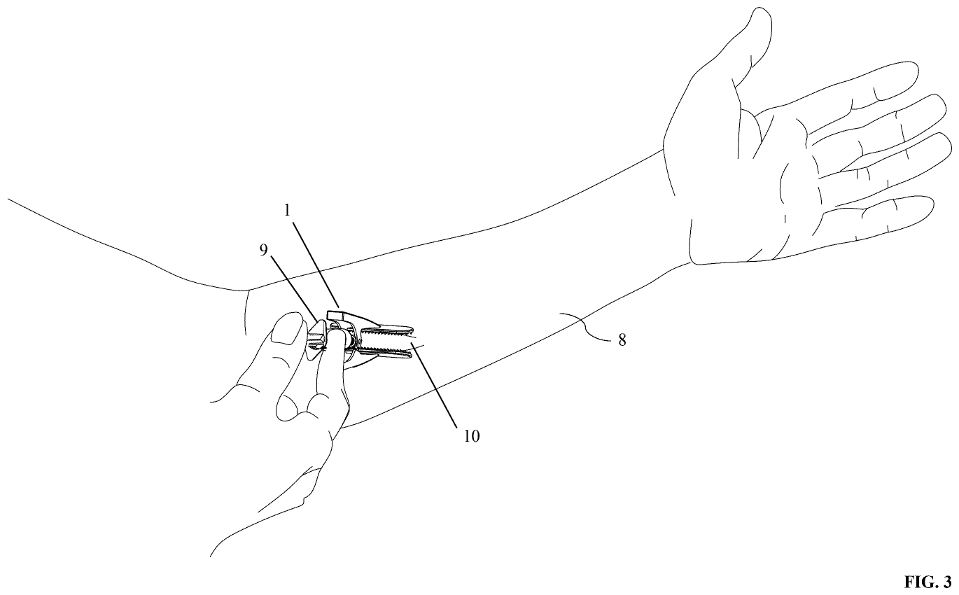

On the other hand, FIG. 3 builds upon the scenario depicted in FIG. 2, illustrating the actual process of administering a subcutaneous injection using the skin-pinching device 1. In this figure, a needle 9 is introduced, shown penetrating the pinched portion of the skin 10 on the hand 8.

The skin-pinching device 1 maintains its secure grip on the targeted skin area, having created a raised, pinched portion of the skin 10. This pinched skin provides a stable and easily accessible site for needle insertion. The space between the first and second pinching ends of the device allows for the needle 9 to be comfortably positioned and guided into the skin at the appropriate angle.

As the needle 9 penetrates the pinched portion of the skin 10, the desensitizing effect of the spikes on the pinching ends becomes apparent. The localized pressure and stimulation provided by the spikes help to minimize the perception of pain or discomfort associated with the needle insertion, making the injection process more tolerable for the user. The needle 9 is depicted as a slender, pointed instrument, typical of those used for subcutaneous injections.

In some aspects, the FIG. 3 effectively showcases the culmination of the skin-pinching device's function, demonstrating its ability to facilitate a more comfortable and precise subcutaneous injection. The device's secure grip on the skin, combined with the desensitizing action of the spikes and the accessibility provided by the space between the pinching ends, contributes to an improved injection experience for the user.

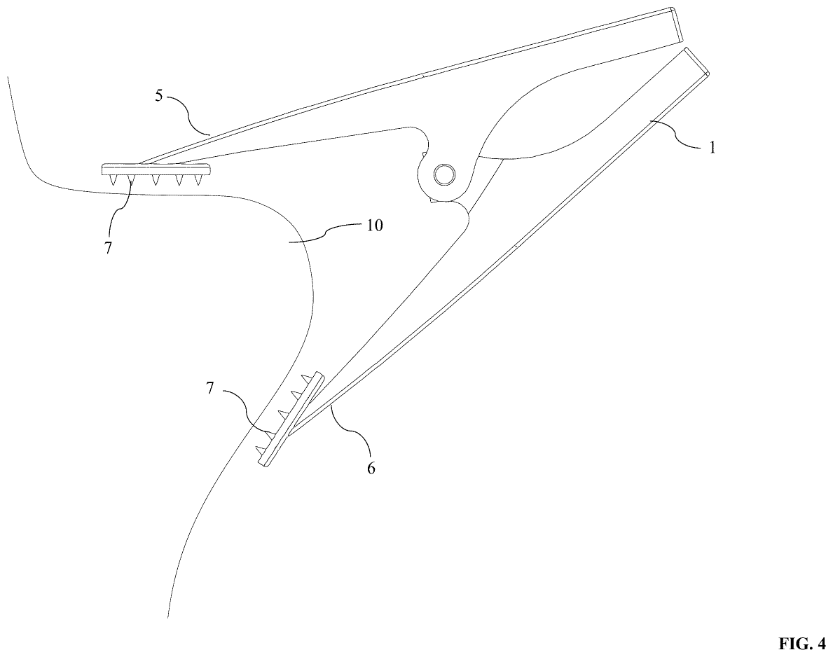

The FIG. 4 provides a close-up view of the skin-pinching device 1 as it attaches to the skin, offering a more detailed look at the interaction between the device and the targeted skin area. The figure highlights the device's ability to raise and isolate a specific section of skin for subcutaneous injection.

In this close-up view, the first pinching end 5 and second pinching end 6 of the device are shown firmly grasping the skin, creating a raised, pinched portion of the skin 10 between them. The pinching action of the device elevates the skin, making it more accessible for needle insertion and ensuring that the injection is delivered into the subcutaneous layer.

The spikes on the first and second pinching ends are visible in this figure, demonstrating their direct contact with the pinched portion of the skin 10. As the spikes apply pressure to the skin, they create localized points of stimulation, which help to desensitize the area and reduce the sensation of the impending injection. The close-up perspective emphasizes the role of the spikes in providing a comfortable injection experience for the user.

The raised, pinched portion of the skin 10 is depicted as a small, elevated area that is clearly defined by the grip of the skin-pinching device 1. The device's pinching action creates a taut and stable surface for needle insertion, minimizing the risk of the needle slipping or missing the intended injection site.

The close-up view in FIG. 4 also highlights the ergonomic design of the skin-pinching device 1. The first pinching end 5 and second pinching end 6 are shown as smooth, curved surfaces that conform to the natural contours of the skin, ensuring a secure and comfortable grip on the targeted area.

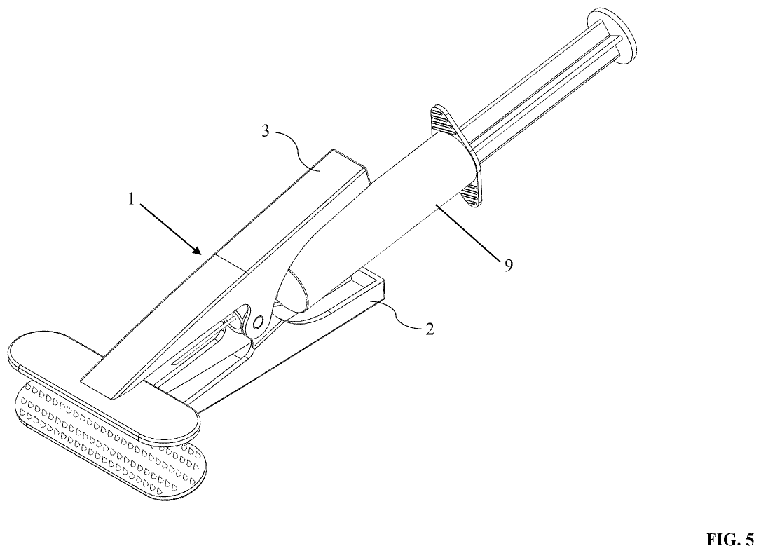

Further, FIG. 5 illustrates the interaction between the skin-pinching device 1 and a syringe with a needle 9 during the subcutaneous injection process. The focus of this figure is on the passage of the syringe's needle between the first handle 2 and second handle 3 of the pinching device.

In the primary embodiment shown, the needle 9 is able to pass unobstructed through the space between the first handle 2 and second handle 3. This space is intentionally designed to provide sufficient clearance for the needle, allowing the user to easily position and insert the needle into the raised, pinched portion of the skin held by the device.

However, the present invention may also incorporate alternative designs and modifications to the skin-pinching device 1 that facilitate needle access and passage. One such modification involves shaping the first handle 2 and second handle 3 to create a C-section or curved opening between them. This C-shaped space would allow the needle 9 to pass through more easily, providing a guided path for needle insertion and reducing the risk of accidental contact with the handles.

Another foreseeable modification is the use of substantially elongated first pinching end 5 and second pinching end 6. By extending the length of the pinching ends, the device would create a larger gap between the handles, allowing ample room for the needle 9 to pass through without obstruction. This design modification would be particularly beneficial for users with larger hands or those requiring more space to maneuver the syringe and needle comfortably.

Additional modifications to the skin-pinching device 1 could include adjustable handles that can be positioned to accommodate different needle sizes or injection angles. This adaptability would allow the device to be customized to suit individual user preferences and specific injection requirements.

Furthermore, the material and construction of the handles could be modified to improve grip, comfort, and stability during the injection process. For example, the handles could incorporate ergonomic contours, textured surfaces, or soft, non-slip materials to enhance user control and reduce hand fatigue.

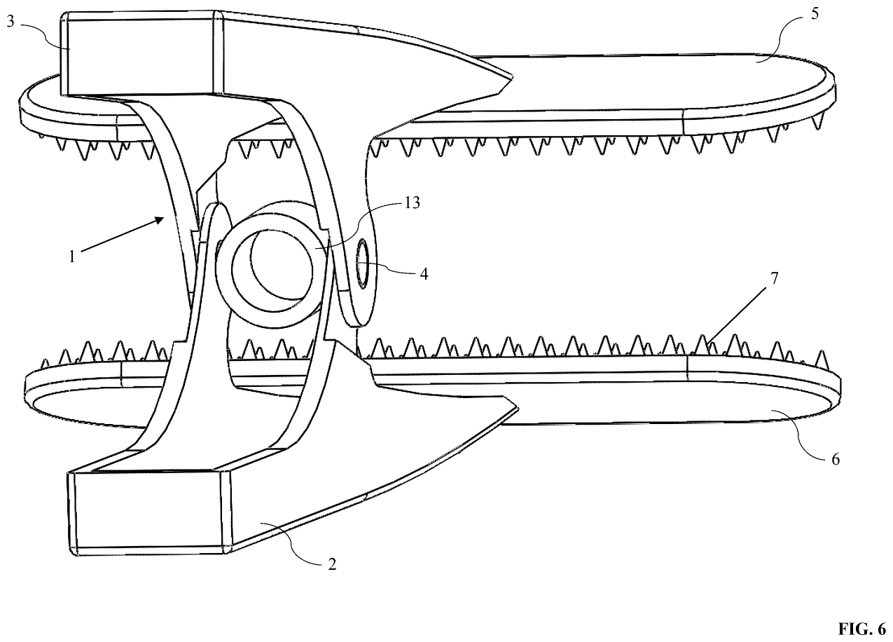

FIG. 6 provides a close-up top view of the skin-pinching device 1 in an open position, offering a preferable embodiment of the device's internal mechanism and components. This exemplary illustration explores the device's spring-loaded design, which plays a crucial role in keeping the first pinching end 5 and second pinching end 6 close together when in use or not in use.

In preferable aspects, the device incorporates a spring mechanism 13, which may be positioned between the first handle 2 and second handle 3, near the pivot point 4 of the device. The spring functions to provide a constant, inward force that keeps the first pinching end 5 and second pinching end 6 pressed together when the handles are not being actively squeezed.

The first pinching end 5 and second pinching end 6 are shown in an open position, revealing their inner surfaces. In this view, the spikes 7 on the pinching ends are clearly visible, arranged in a pattern optimized for gripping and desensitizing the skin. The spikes 7 are shown as small, pointed protrusions that extend inwardly from the pinching ends, ready to engage with the skin when the device is closed.

The close-up top view also highlights the precise alignment and symmetry of the first pinching end 5 and second pinching end 6. The pinching ends are designed to meet evenly and accurately when the device is closed, ensuring a secure and uniform grip on the skin. The gap between the pinching ends in the open position is carefully calibrated to allow the device to be easily positioned over the targeted skin area before closing.

The spring may be in an expanded state, exerting an outward force on the first handle 2 and second handle 3. This outward force may be balanced by the device's pivot point 4, which allows the handles to rotate smoothly and maintain the proper alignment of the pinching ends. The spring's strength is carefully selected to provide sufficient closing force to grip the skin securely, while still allowing the user to easily open the device by squeezing the handles together.

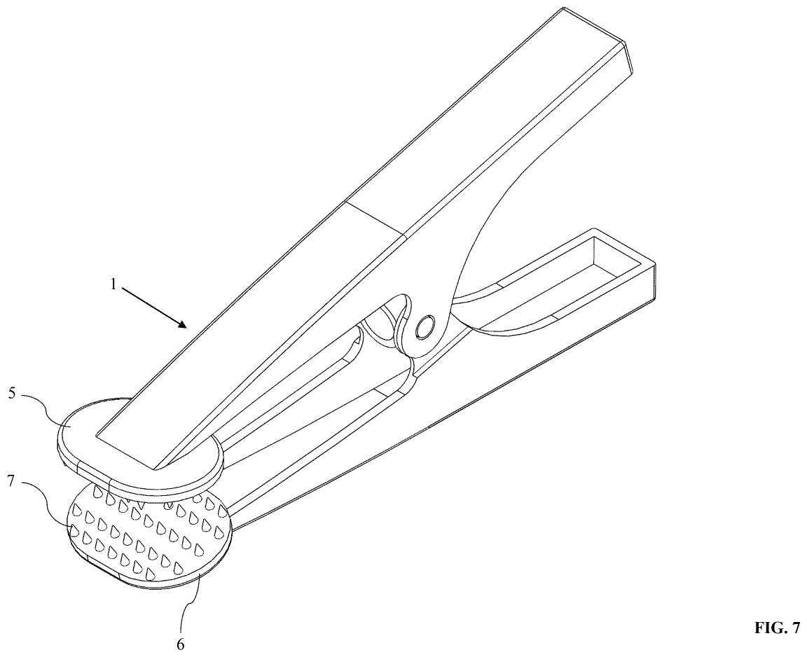

The FIG. 7 provides a view of an alternative design for the first pinching end 5 and second pinching end 6 of the skin-pinching device 1. In this embodiment, the pinching ends feature a rounded construction, which offers several potential benefits for user comfort and skin engagement.

The rounded pinching ends may be characterized by smooth, curved surfaces that transition from the base of the pinching end to the tip. This rounded construction eliminates any sharp edges or abrupt angles, creating a gentler and more ergonomic interface between the device and the user's skin. The smooth contours of the pinching ends help to distribute the gripping force more evenly across the skin surface, reducing the risk of localized pressure points or discomfort.

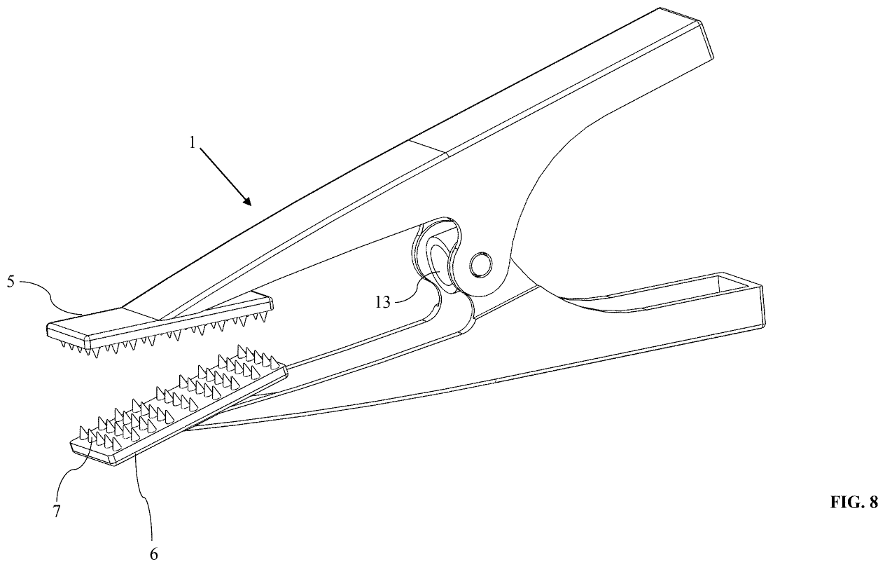

The FIG. 8 illustrates another alternative design for the first pinching end 5 and second pinching end 6 of the skin-pinching device 1. In contrast to the rounded construction shown in FIG. 7, this embodiment features a substantially squared construction of the pinching ends, which offers its own set of potential advantages.

The squared pinching ends may be characterized by flat, perpendicular surfaces that create a more angular and defined shape. The edges of the square construction are crisp and well-defined, providing a clear boundary between the pinching surfaces and the surrounding area. This square design may be beneficial in certain scenarios where a more precise and controlled grip on the skin is required.

The spikes 7 on the square pinching ends may be arranged in a grid-like pattern, with each spike positioned at a specific intersection point on the flat surface. This ordered arrangement may allow for a uniform distribution of the spikes across the pinching area, ensuring consistent skin engagement and desensitization. The square construction may also facilitate the manufacturing process, as the flat surfaces and precise spike placement could be easier to produce and replicate consistently.

In some aspects, the skin-pinching device 1 may incorporate adjustable tension settings for the spring 13. This feature would allow users to customize the gripping force of the device to their individual preferences and skin sensitivity levels. By adjusting the spring tension, users can find the optimal balance between secure skin engagement and comfort, ensuring a personalized and effective injection experience.

In some aspects, the first pinching end 5 and second pinching end 6 may be coated with a soft, cushioning material, such as silicone or rubber. This coating would provide an additional layer of comfort and protection for the user's skin, reducing the risk of irritation or abrasion caused by the pinching action. The soft coating may also enhance the device's grip on the skin, preventing slippage and ensuring a stable platform for needle insertion.

In some aspects, the skin-pinching device 1 may include a built-in numbing agent dispenser. This feature would allow users to apply a topical anesthetic directly to the injection site before using the device. The numbing agent would work in conjunction with the desensitizing action of the spikes 7 to further minimize any pain or discomfort associated with the injection process. The dispenser could be conveniently integrated into the device's handles or pinching ends, providing a streamlined and efficient numbing solution.

In some aspects, the spikes 7 on the first pinching end 5 and second pinching end 6 may be made from a flexible or retractable material. This design modification would allow the spikes to adapt to the contours of the skin more effectively, providing a gentler and more comfortable gripping experience. Flexible or retractable spikes could also reduce the risk of skin damage or puncture, as they would be less likely to apply excessive localized pressure.

In some aspects, the skin-pinching device 1 may incorporate a vibration function to further desensitize the injection site. The device could include a small, integrated vibration motor that activates when the pinching ends engage with the skin. The vibration would create an additional sensory distraction, potentially reducing the perception of pain or discomfort during the injection process. This feature could be particularly beneficial for users with needle anxiety or heightened skin sensitivity.

In some aspects, the device may include a built-in LED light to illuminate the injection site. This feature would enhance visibility and accuracy when positioning the device and inserting the needle, particularly in low-light environments or when injecting in hard-to-see areas of the body. The LED light could be strategically placed near the pinching ends or integrated into the handles, providing targeted illumination exactly where it is needed.

### INDUSTRIAL APPLICATION

The skin-pinching device for facilitating subcutaneous injections has widespread industrial applicability in the medical and healthcare sectors. The device is particularly useful for patients requiring frequent subcutaneous injections, such as those with diabetes, hormonal disorders, or allergies. It can be utilized in various settings, including hospitals, clinics, and home healthcare environments. The device's unique design and features, such as the spiked pinching ends and ergonomic handles, make it an attractive option for healthcare professionals and patients seeking a more efficient and comfortable injection experience. The invention has the potential to improve patient compliance, reduce injection-related anxiety, and enhance the overall quality of care in the administration of subcutaneous medications.

What is claimed is:

1. A skin-pinching device for facilitating subcutaneous injections, comprising:
a first handle and a second handle connected at a pivot;
a first pinching end pivotally connected to the first handle and a second pinching end pivotally connected to the second handle, wherein the first and second pinching ends each comprise a plurality of spikes;
wherein when the first and second handles are pressed together, the first and second pinching ends move away from each other, allowing the device to be placed over a section of skin to be pinched:
wherein when the first and second handles are released, the first and second pinching ends move towards each other, pressing against the skin to securely grip the section of tissue, enabling the user to administer a subcutaneous injection using one hand while the device holds the pinched skin in place, and
wherein the first and second handles are positioned to provide sufficient space for needle insertion between the first and second pinching ends when the handles are squeezed together.

2. The skin-pinching device of claim 1, wherein the plurality of spikes are coated with a high-friction material to enhance grip on the skin.

3. The skin-pinching device of claim 2, wherein the high-friction material is selected from the group consisting of rubber and polymers.

4. The skin-pinching device of claim 1, wherein the first and second pinching ends each comprise a substantially flattened section for gripping the skin.

5. The skin-pinching device of claim 1, wherein the pivot comprises a mechanism for self-closing the first and second pinching ends when the first and second handles are released.

6. The skin-pinching device of claim 1, wherein the pivot comprises a substantially enlarged through hole between the first and second handles to allow passage of a needle.

7. The skin-pinching device of claim 1, wherein the pivot is substantially curved between the first and second handles, forming a C-shape to allow passage of a needle.

8. The skin-pinching device of claim 1, wherein the first and second pinching ends are detachable and replaceable, allowing for easy cleaning or replacement of the spiked surfaces.

9. The skin-pinching device of claim 1, further comprising an LED light positioned to illuminate the pinched portion of skin, thereby improving visibility of the injection site in low-light conditions.

10. The skin-pinching device of claim 1, further comprising a built-in dispenser for a numbing gel, wherein the dispenser is configured to apply the numbing gel directly to the skin prior to pinching and injecting.

11. The skin-pinching device of claim 1, wherein the space between the first and second pinching ends is configured to allow needle insertion into the pinched portion of skin from either side of the device when the skin is gripped.

12. A method for administering a subcutaneous injection using a skin-pinching device, the method comprising:
providing a skin-pinching device comprising:
a first handle and a second handle connected at a pivot;
a first pinching end pivotally connected to the first handle and a second pinching end pivotally connected to the second handle, wherein the first and second pinching ends each comprise a plurality of spikes;
pressing the first and second handles together, causing the first and second pinching ends to move away from each other;
placing the device over a section of skin to be pinched;
releasing the first and second handles, causing the first and second pinching ends to move towards each other, pressing against the skin to securely grip the section of tissue; and
administering a subcutaneous injection using one hand while the device holds the pinched skin in place.

13. The method of claim 12, wherein the plurality of spikes are coated with a high-friction material to enhance grip on the skin.

14. The method of claim 13, wherein the high-friction material is selected from the group consisting of rubber and polymers.

15. The method of claim 12, wherein the first and second pinching ends each comprise a substantially flattened section for gripping the skin.

16. The method of claim 12, wherein the pivot comprises a mechanism for self-closing the first and second pinching ends when the first and second handles are released.

17. The method of claim 12, wherein the pivot comprises a substantially enlarged through hole between the first and second handles to allow passage of a needle.

18. The method of claim 12, wherein the pivot is substantially curved between the first and second handles, forming a C-shape to allow passage of a needle.

19. The method of claim 12, wherein the first and second pinching ends are detachable and replaceable, allowing for easy cleaning or replacement of the spiked surfaces.
